(19) United States
(12) Patent Application Publication
(10) Pub. No.: US 2024/0369663 A1
(43) Pub. Date: Nov. 7, 2024

(54) MOTION CORRECTION FOR SPATIOTEMPORAL TIME-RESOLVED MAGNETIC RESONANCE IMAGING

(71) Applicant: The General Hospital Corporation, Boston, MA (US)

(72) Inventors: Kawin Setsompop, Stanford, CA (US); Zijing Dong, Cambridge, MA (US); Fuyixue Wang, Cambridge, MA (US)

(21) Appl. No.: 18/552,745

(22) PCT Filed: Mar. 28, 2022
(86) PCT No.: PCT/US2022/022125

§ 371 Date: Sep. 27, 2023

Related U.S. Application Data
(60) Provisional application No. 63/167,090, filed on Mar. 28, 2021.

(51) Int. Cl. G01R 33/565; G01R 33/24; G01R 33/48; G01R 33/56; G01R 33/561

(52) U.S. Cl. CPC G01R 33/56509; G01R 33/243; G01R 33/4818; G01R 33/5602; G01R 33/5616; G01R 33/56563

(57) ABSTRACT

Motion correction in spatiotemporal time-resolving magnetic resonance imaging (“MRI”) include a motion estimation component and a motion correction component. The motion estimation component can include a spatiotemporal time-resolved data acquisition that is configured to acquire navigator data in order to obtain motion parameters and estimate changed in B₀ inhomogeneity caused by subject motion. Motion-corrected reconstruction can be used to recover accurate motion-corrected images by modeling the motion into the reconstruction. A subspace reconstruction framework can be used for both navigator reconstruction when estimating motion parameters, and for reconstructing the motion-corrected images.

### STATEMENT OF FEDERALLY SPONSORED RESEARCH

This invention was made with government support under EB020613, MH116173, and EB025162 awarded by the National Institutes of Health. The government has certain rights in the invention.

### BACKGROUND

Magnetic resonance imaging (“MRI”) is a non-invasive imaging method that has been widely used in both clinical diagnosis and neuroscience research. A major advantage of MRI is that it can generate different image contrasts and measure various tissue properties by using different types of MR sequence. In order to obtain rich information to assess tissues, multiple contrasts (e.g., T1-weighted images, T2-weighted images, FLAIR) are normally acquired in clinical practice. A more quantitative approach to obtain these information is multiparametric mapping that can quantitatively measure the tissue properties, such as relaxation rates and magnetic transfer effect. However, the acquisition of both multi-contrast imaging and multiparametric mapping usually requires long scan time. To achieve fast imaging, spatiotemporal acquisition and time-resolving approaches have been developed recently. The main idea of spatiotemporal and time-resolving methods is in designing MR sequence and encoding to acquire MR signals with different contrast weighting and spatial encoding, and then estimate the multi-contrast images and/or quantitative parameters from the signals by image reconstruction.

For example, echo-planar time-resolved imaging (“EPTI”) is a spatiotemporal time-resolving technique that can obtain thousands of multi-contrast images that are resolved from the signals acquired with high spatiotemporal correlation. In addition, multiple quantitative parameters can also be calculated using these multi-contrast images.

The spatiotemporal time-resolving techniques provide more efficient acquisition for multi-contrast and quantitative mapping, however, the image quality and accuracy can be significantly compromised by the subject movements during the scan, which is a common challenge for MRI acquisition. Hence, the development of motion correction methods is helpful to make spatiotemporal time-resolving techniques robust to subject motion for clinical application, especially for low-compliance patients (e.g., pediatric patients).

### SUMMARY OF THE DISCLOSURE

The present disclosure addresses the aforementioned drawbacks by providing a method for magnetic resonance imaging. The method includes accessing magnetic resonance data acquired with a magnetic resonance imaging (MRI) system, wherein the magnetic resonance data include spatiotemporally acquired data and navigator data. Motion data are estimated from the navigator data using a computer system, wherein the motion data include motion parameters associated with subject motion that occurred when the magnetic resonance data were acquired and B₀ change data that indicate B₀ inhomogeneity changes caused by the subject motion. An image is reconstructed from the spatiotemporally acquired data using a subspace reconstruction framework that models motion using the motion data, where the image has reduced motion artifacts.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates a self-navigator acquisition for spatiotemporal methods, in which a three-contrast acquisition with three repetition times (“TRs”) is illustrated as an example. Two different acquisition strategies are shown. The acquisition order without self-navigator acquires three contrasts at the same block in every TR, and only provides central k-space signals at the first TR. The self-navigated acquisition order acquires three contrasts at different blocks and provides a central k-space block sampling in every TR to estimate motion parameters between different TRs.

FIG. 2A illustrates an example spatiotemporal data acquisition implemented as an inversion recovery 3D echo planar time-resolved imaging (“EPTI”) acquisition with navigator data acquired during a magnetization recovery period.

FIG. 2B illustrates an example optimized spatiotemporal encoding pattern that can be employed in the navigator acquisition for large k-t block sampling after each excitation. In addition, a partial-Fourier scheme was used to acquire the navigator, to allow a larger coverage along k_{y} and k_{z} after reconstruction with less excitations (4 excitations in this example).

FIG. 2C illustrates different block patterns for 4D navigator acquisitions. An asymmetric partial-Fourier pattern (Case 3) provides good estimation close to the 9-block acquisition (Case 6), but with only 4 blocks, corresponding to much less time and fewer excitations for the 4D navigator acquisition.

FIG. 3 is a flowchart setting forth the steps of an example method for motion estimation and correction of spatiotemporal acquisitions in accordance with some embodiments described in the present disclosure.

FIG. 4 is a flowchart setting forth the steps of an example method for motion estimation from navigator data in accordance with some embodiments described in the present disclosure.

FIG. 5 is a block diagram of an example magnetic resonance imaging (“MRI”) system that can implement some embodiments described in the present disclosure.

FIG. 6 is a block diagram of an example system for estimating motion and reconstructing motion-corrected images in accordance with some embodiments described in the present disclosure.

FIG. 7 is a block diagram of example components that can implement the system of FIG. 6.

### DETAILED DESCRIPTION

Described here are systems and methods for motion correction in spatiotemporal time-resolving magnetic resonance imaging (“MRI”). The motion correction techniques described in the present disclosure are capable of achieving motion-robust acquisitions and ensuring good image quality even with subject motion during scan. In general, the systems and methods include two components: a motion estimation component and a motion correction component. As a non-limiting example, the motion estimation component can include a spatiotemporal time-resolving data acquisition that is configured to acquire navigator data in order to obtain the motion parameters. For motion correction, as a non-limiting example, a retrospective motion-corrected reconstruction can be used to recover accurate motion-corrected images by modeling the motion into the reconstruction. It will be appreciated by those skilled in the art that the motion estimation and motion correction components described in the present disclosure can also be combined with other techniques for motion-robust spatiotemporal time-resolving imaging. For example, existing prospective motion correction techniques can be combined with the proposed motion estimation method to correct the applied gradients in real time.

In spatiotemporal time-resolving techniques, different sections of k-space can be acquired with different contrast weightings across time. By designing the pulse sequence to acquire the central part of the k-space signals at different times across the whole acquisition (e.g., every repetition time (“TR”) or every couple of seconds), a low-resolution navigator image and/or volume can be obtained for motion estimation. The acquisition of these navigator data can be implemented as self-navigation that uses the signals acquired by the original spatiotemporal time-resolving sequence, as an extra-navigation that adds a short additional navigator acquisition to the original sequence, or the like.

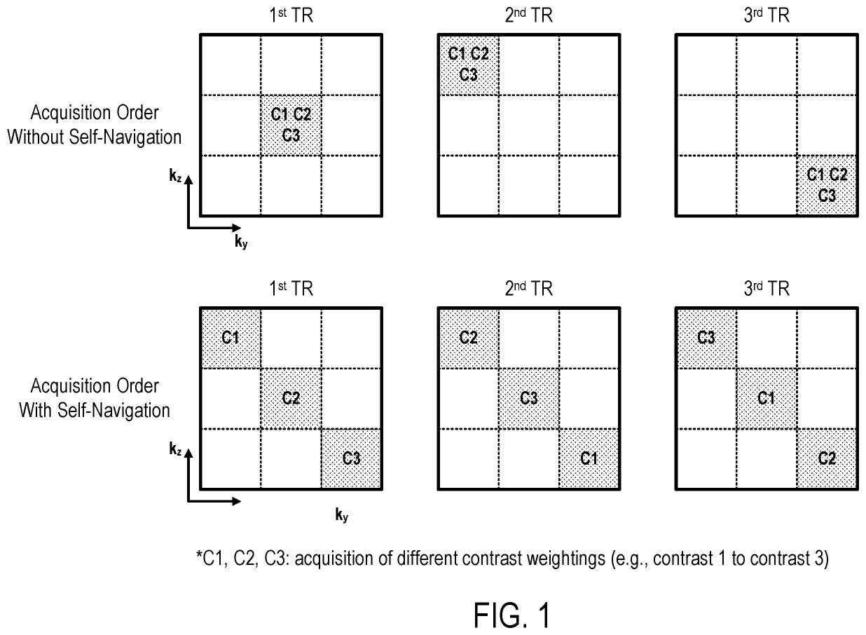

As a general example, a three-contrast acquisition with three TRs can be used to implement the proposed navigator acquisition, as illustrated in FIG. 1. In FIG. 1, the two different acquisition strategies (with and without self-navigation) are designed to acquire the same signals in k-space (three contrasts for three blocks) by three TRs. The acquisition order without self-navigation acquires the three contrasts at the same block in every TR, and only provides central k-space signals at the first TR. The self-navigated order acquires different contrasts at different blocks and provides a central k-space block sampling in every TR. The signals from the central k-space block can be used as a navigator to estimation motion parameters between different TRs.

Using the central k-space signals for self-navigation, relative motion parameters between the navigators across TRs can be estimated, which represents the motion between signals acquired at different times. The estimation can be performed in k-space directly or in the image domain using registration algorithms.

One example of spatiotemporal time-resolving pulse sequences that can be implemented with the systems and methods described in the present disclosure are echo planar time-resolved imaging (“EPTI”) pulse sequences, such as the ones described in co-pending U.S. Pat. No. 11,022,665, which is herein incorporated by reference in its entirety. EPTI pulse sequences are capable of resolving hundreds of distortion and blurring free images across a modified continuous echo-planar imaging (“EPI”) readout to track signal evolution. The continuous data sampling with bipolar readout after each excitation provides high acquisition efficiency, and the densely acquired signals with strong spatiotemporal correlation enables the design of highly-accelerated encoding in the spatiotemporal domain. By exploiting both the temporal signal correlation and the spatial information from multi-channel receiver coils, the full k-t (frequency-echo) signals can be well recovered from highly-undersampled EPTI data, resolving hundreds or thousands of distortion and blurring-free multi-contrast images. EPTI pulse sequences can be implemented for two-dimensional (“2D”) or three-dimensional (“3D”) acquisitions. 3D-EPTI pulse sequences can enable ultra-fast acquisition of multiple quantitative parameters at isotropic resolution under higher acceleration rates by taking advantage of the spatiotemporal correlation within and between the readouts with optimized encoding in the four-dimensional (“4D”) k-t space (i.e., kₓ-k_{y}-k_{z}-t space).

Thus, in some embodiments, a 3D-EPTI pulse sequence is modified to include an efficient 4D navigator acquisition to achieve fast and motion-robust quantitative imaging with whole-brain coverage. The 4D navigator (x-y-z-echoes) can be designed to estimate both 3D rigid motion (e.g., 6 degrees-of-freedom (“DOFs”)) and the B₀-inhomogeneity change (ΔB₀) caused by subject motion (e.g., head movements) in every TR. To avoid extra scan time, the navigator can be acquired during the deadtime for magnetization recovery of the sequence. An optimized highly-undersampled spatiotemporal encoding combined with a partial-Fourier scheme can be utilized to achieve large k-t space data coverage with only four small flip-angle (“FA”) excitations and readouts, which can reduce the SNR cost of the navigator acquisition to less than 1% based on simulation analysis.

By modeling the motion and ΔB₀ of every TR in a motion-corrected 3D subspace reconstruction, multi-contrast images with reduced motion artifacts can be recovered for quantitative fitting. In some implementations, an inversion-recovery gradient-echo (“IR-GE”) EPTI sequence can be used for simultaneous T₁ and T₂* mapping, which provides for motion-robust quantitative imaging.

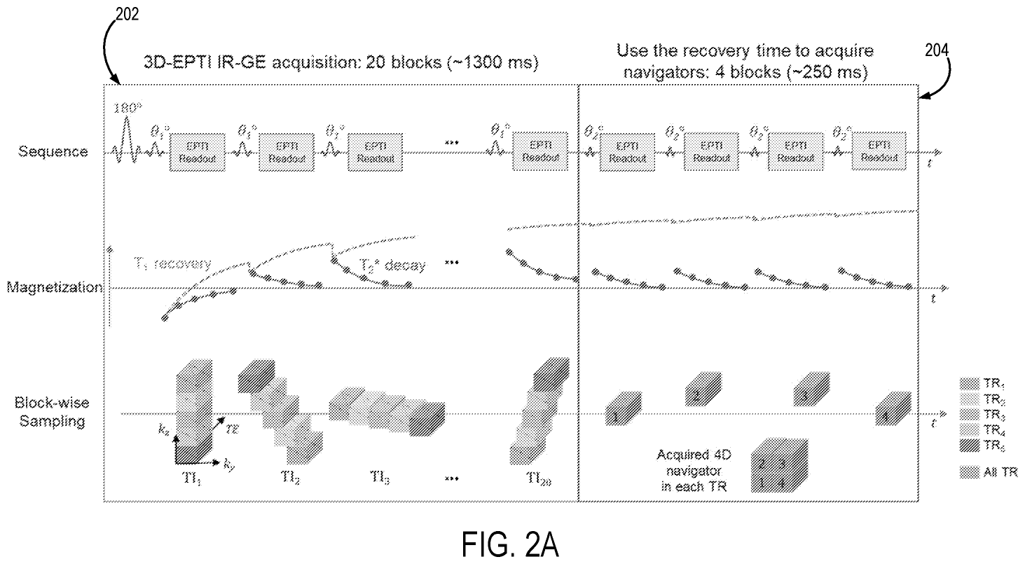

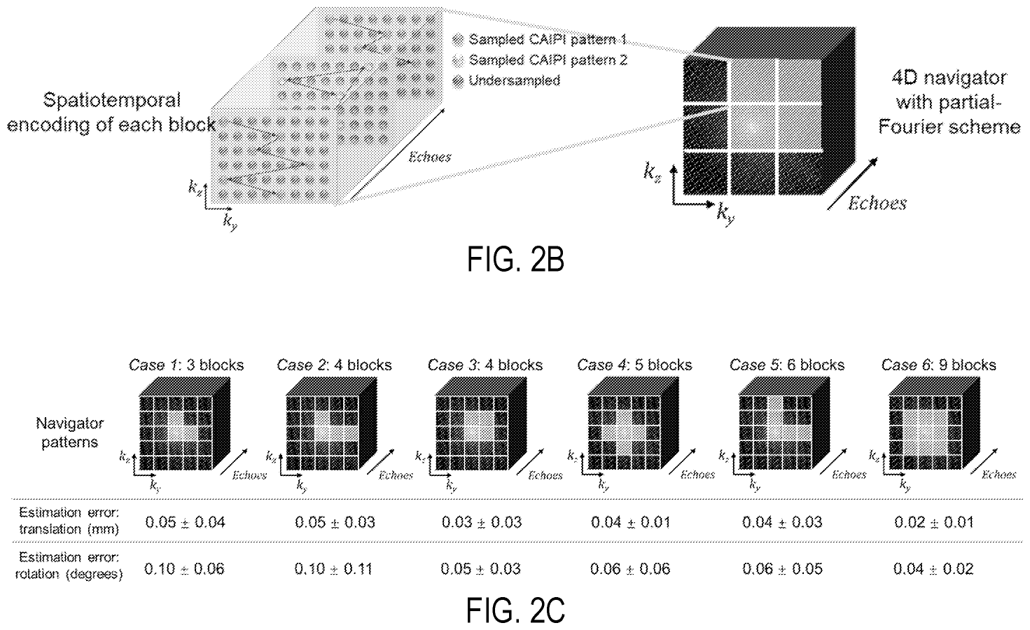

An example of a 3D-EPTI pulse sequence is illustrated in FIG. 2A. In this particular example, the 3D-EPTI pulse sequence is implemented as an IR-GE 3D-EPTI pulse sequence. The IR-GE 3D-EPTI pulse sequence includes an acquisition period 202 and a recovery period 204. During the acquisition period 202, multiple excitation pulses are applied after an inversion pulse with 3D-EPTI readouts to track the T₁ recovery and T₂* decay. Each 3D-EPTI readout utilizes a continuous sampling to acquire multiple time points with different k_{y}-k_{z} encoding to cover a large 4D block in the k-t space, as illustrated in FIG. 2B.

An optimized spatiotemporal CAIPI (controlled aliasing in parallel imaging) encoding scheme can be employed to provide accurate image reconstruction under high undersampling factors (e.g., 80×) by exploiting strong temporal signal correlations across the EPTI readout and additional multi-channel coil information with a complementary pattern. In order to further accelerate the acquisition, 3D-EPTI can acquire a radial-block pattern at each inversion time (“TI”) with fewer TRs instead of the full k-t space, which creates incoherent aliasing across TIs to allow image recovery (e.g., based on compressed sensing-based techniques) by exploiting the temporal correlation between these readouts. By combining the spatiotemporal CAIPI encoding and radial block sampling, high-quality quantitative imaging can be acquired at a high undersampling rate in k-t space and reconstructed with a low-rank subspace reconstruction approach. For example, whole-brain quantitative imaging at 1-mm isotropic resolution can be acquired in two minutes, with a 4D block size of 8×10×210×48 (k_{y}×k_{z}×kₓ×nEcho) and 45 TRs to form two radial-block blades at each TI (assuming TR=2.6 s).

At the end of each TR in the IR-GE EPTI acquisition, there is recovery period 204 during which no excitations and/or readouts are typically applied in order to allow for recovery of the longitudinal magnetization (M_{z}) before the next TR. For example, the recovery period 204 may have a duration of around 500-1000 ms. To avoid any increase in the overall data acquisition time, navigator data can be acquired during this recovery period 204.

In general, the navigator is designed to have minimal effect on the desired M_{z} recovery process. For instance, the navigator can be designed using an optimized temporal-variant spatiotemporal encoding (e.g., the temporal-variant spatiotemporal CAIPI encoding shown in FIG. 2B) to cover a large 4D block after each excitation. As another example, the navigator data can be acquired using a partial-Fourier scheme along both k_{y} and k_{z}, as also shown in FIG. 2B (right), which allows for a recovery of higher frequency signals with fewer excitations. As yet another example, a smaller flip angle than what is used in the image acquisition (e.g., 02<01) can also be utilized to further reduce the impact of the extra excitations on M_{z}.

These optimizations of the navigator data acquisition not only reduce the cost of the signal recovery and SNR efficiency, but also keep the differences in the M_{xy} signal generated by these small-FA excitations at the end of the inversion process to be relatively small; thus, the 4D blocks acquired at different TIs with different k_{y}-k_{z} encodings can be combined along TI with reduced timepoints to reconstruct (as shown in FIG. 2A, 4 TI-blocks can be combined to form a navigator).

To provide accurate estimation with fewer excitations, different block pattern schemes can be implemented, such as those shown in FIG. 2C. In the examples shown in FIG. 2C, the use of a partial-Fourier scheme can be seen as providing accurate 3D motion estimation with much fewer excitations. As a non-limiting example, four excitations can be utilized to acquire the 4D navigator acquisition (e.g., as shown in FIG. 2A), which in some implementations can take approximately 250 ms during the signal recovery period 204 in each TR, allowing a k-t space coverage of 30× 24× 150× 48 (k_{y}× k_{z}× kₓ×1), corresponding to a resolution of ˜7× 7×1.5 mm³ and a 40 ms readout, for accurate motion and ΔB₀ estimation.

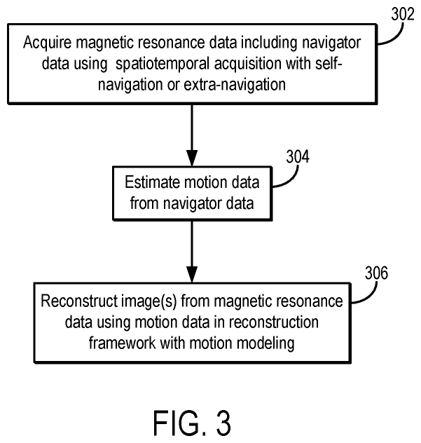

The general motion correction framework using the proposed estimation and correction approach is shown in FIG. 3. The method includes accessing magnetic resonance data with a computer system, where the magnetic resonance data (e.g., k-space data, k-t space data) are acquired using a spatiotemporal acquisition and include navigator data acquired with self-navigation or extra-navigation, as indicated at step 302. As described above, a time-resolved data acquisition such as an EPTI acquisition can be implemented. In some embodiments, the navigator data can be acquired during the longitudinal magnetization recovery period.

The magnetic resonance data can be accessed by retrieving previously acquired data from a memory or other data storage device or medium. Additionally or alternatively, the data can be accessed by acquiring the data with an MRI system and communicating the acquired data to the computer system, which may be a part of the MRI system.

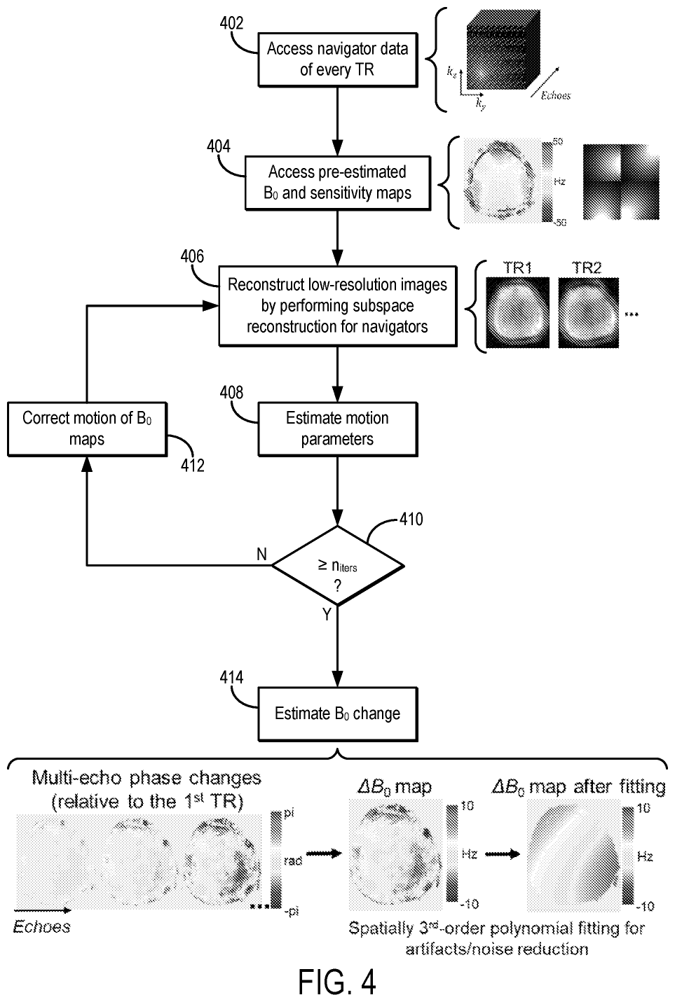

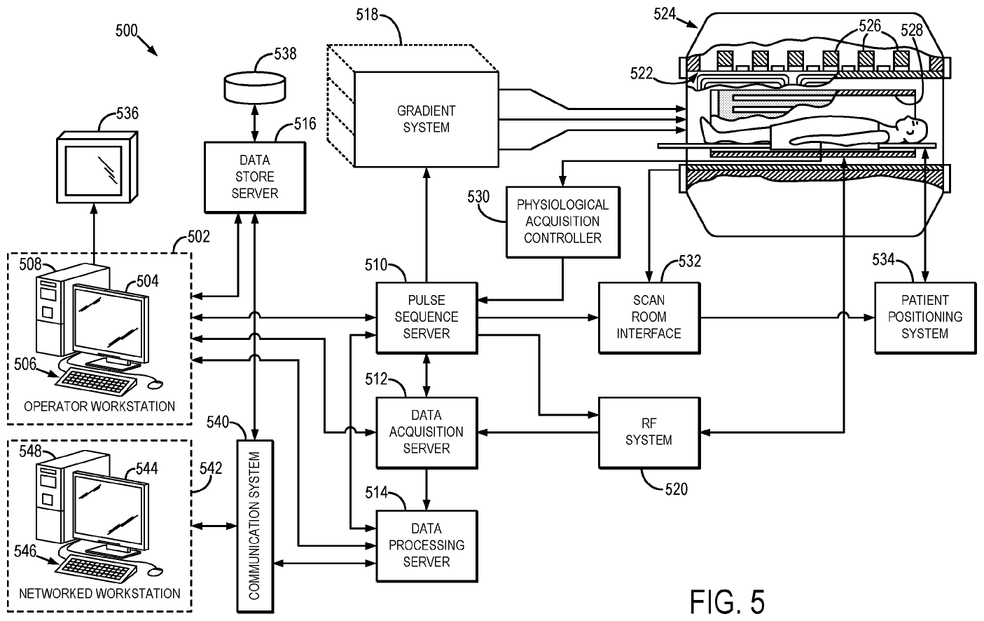

Motion data are then estimated from the navigator data, as indicated at step 304. For instance, the navigator data can be reconstructed and motion data estimated from the reconstructed navigator data. As a non-limiting example, the navigator data can be reconstructed using a subspace reconstruction. In some embodiments, the motion data include both an estimate of subject motion and also changes in B₀ caused by the motion (ΔB₀). An example method for estimating motion data is described below in more detail with respect to FIG. 4.

After the motion data are estimated, images are reconstructed from the acquired magnetic resonance data using a reconstruction framework that incorporates the estimated motion data into a motion modeling, as indicated at step 306. For example, using the estimated motion and ΔB₀ parameters in the motion data estimated from the navigators, a motion-and-phase corrected 3D subspace reconstruction can be implemented to recover multi-contrast volumes acquired by the spatiotemporal pulse sequence with reduced motion artifacts. As a non-limiting example, to reconstruct images without motion artifacts from data acquired using a spatiotemporal acquisition, the motion can be modeled during the reconstruction process. In general, motion between acquired signals can be modeled using estimated parameters and added into the reconstruction of the spatiotemporal signals. For instance, motion transforms can be modeled in a subspace reconstruction to reconstruct multi-contrast images as:

$${min}_{c} {‖ {U}_{n} F S {B}_{n} T ϕ c - {y}_{n} ‖}_{2}^{2} + γ R ( c ) \qquad ( 1 )$$

where ϕ corresponds to the subspace bases; c corresponds to the coefficient maps of the bases; T is the motion transform operator on images (e.g., a rigid motion transformation), which can transform the multi-contrast volumes into N different motion states, where N can be equal to the number of TRs used when acquiring the magnetic resonance data; S is the coil sensitivity; F is the Fourier transform; Uₙ is the undersampling mask for the nth motion state; yₙ is the acquired undersampled k-space or k-t space data for the nth motion state; and Bₙ is the updated phase evolution for the nth motion state using the estimated motion data (e.g., ΔB₀S). The regularization term, R(c), can be incorporated to improve the conditioning, and λ is the control parameter. Here, the motion transform operator, T, ensures the data fidelity will be performed between the acquired signals and the estimate signal(s) with correct motion states, which corrects the modeling error of reconstruction due to motion. By modeling the motion and ΔB₀ of every TR in the 3D subspace reconstruction, the motion artifacts in the reconstructed multi-contrast images can be effectively reduced.

The reconstructed image(s) can then be displayed to a user or stored for later use, as indicated at step 308. For example, the images can be multi-contrast images that are subsequently processed to generate quantitative parameter maps (e.g., T1 maps, T2 maps, etc.) without motion artifacts. Quantitative parameters including T1, T2*, proton density (PD) and B₁⁺ maps can then be estimated by dictionary matching, as an example.

Referring now to FIG. 4, a framework for navigator reconstruction and motion/ΔB₀ estimation is shown. Navigator data are accessed with the computer system, as indicated at step 402. Pre-estimated B₀ and/or sensitivity maps are also accessed, as indicated at step 404.

The navigator data are then reconstructed into multi-echo volumes for every TR, as indicated at step 406. As a non-limiting example, the following objective function can be utilized for reconstructing the multi-echo navigator volumes using a subspace reconstruction:

$$min {‖ UFSB ϕ c - y ‖}_{2}^{2} + λ R ( c ) ; \qquad ( 2 )$$

where ϕ is the temporal subspace bases generated based on the signal model by an extended phase graph (“EPG”)-based simulation, c is the coefficient maps of the subspace bases, B is the pre-calculated phase evolution (resulting from B₀ inhomogeneity) across different echoes, S is the coil sensitivity, F denotes the Fourier transform operator, U is the undersampling mask, and γ is the acquired undersampled 4D navigator data. R (c) is the locally-low-rank (“LLR”) regularization applied on c, and λ is the control parameter. The pre-calculated B₀ and coil sensitivity maps can be obtained from a fast k-t calibration scan. After solving c, the multi-echo images can be obtained by ϕc, with no image distortion. In addition to estimating motion parameters using the reconstructed images, the B₀ changes due to motion can also be estimated using the multi-echo phases. During motion, not only the B₀-inhomogeneity maps will translate/rotate, but the actual local B₀ values would also change (ΔB₀) due to the interaction of shimming field and head position.

In order to accurately estimate the change of B₀, different ΔB₀s ranging from −50 Hz to 50 Hz can be simulated in the subspace basis generation step. The use of the subspace approach with prior information from the signal model for k-t reconstruction can significantly reduce the number of unknowns in the optimization process, leading to reduced image artifacts and improved SNR.

After the initial navigator reconstruction, 3D rigid motion parameters (e.g., 6 DOFs) of every TR can be estimated from the low-resolution volumes using the FLIRT tool from the FSL software (https://fsl.fmrib.ox.ac.uk/fsl/fslwiki/), or another suitable motion estimation method, as indicated at step 408. To estimate the value changes of the B₀ field (ΔB₀), the navigator reconstruction can be iterated to correct the motion (translation and rotation) of the pre-calculated B₀ maps based on the estimated motion parameters in the previous iteration, as indicated at decision block 410 and step 412. This also improves the reconstruction performance since the motion-corrected pre-calculated B₀ map is closer to the field of the acquired data.

After several iterations (e.g., three iterations, or a number of iterations after which the estimation is stable), the final multi-echo low-resolution volumes are reconstructed for motion and ΔB₀ estimation for every TR, as indicated at step 414. The phase changes of the multi-echo volumes can first be calculated by subtracting the phase from the first TR. Temporal linear fitting can then be applied across TEs per TR to obtain a raw ΔB₀ map. A polynomial fitting (e.g., a spatially third order polynomial fitting) can then be applied to the raw ΔB₀ maps to remove potential image artifacts and improve the SNR, by assuming the ΔB₀ should be spatially smooth since it should be caused by the interaction of shimming and subject position (e.g., head position).

In an example study, IR-GE EPTI data were simulated based on the pulse sequence illustrated in FIG. 2A. Two motion types were simulated by applying motion transformations to different TRs, including random motion and sudden motion (within +8 mm/degrees). 2-mm isotropic datasets were simulated, with a matrix size of 110× 88× 70× 48× 24 (k_{y}× k_{z}× kₓ×nEcho×nTI), and the last 4 TIs were the navigator acquisition. The flip angle for gradient echo excitations was 0=30°, and 02=10° for navigators, echo spacing (“ESP”)=0.7 ms, TR=2.1 s, and 23 TRs were simulated to form a 2 radial-block line acquisition. The simulated data were undersampled using the 3D-EPTI spatiotemporal sampling pattern (block size=10× 8, k_{y}× k_{z}), and 32-channel data are generated with added noise in the k-t space (SNR=20 based on the L2 norm between k-space signal and noise). The 4D navigator data were also generated for each TR using the 4-block pattern as shown in FIG. 2B.

In order to quantitatively evaluate the proposed motion estimation and correction in-vivo, motion-free IR-GE 3D-EPTI datasets at 3 different head positions were acquired with independent pre-calibration scans, and these datasets were subsampled and combined together afterwards to mimic a motion-corrupted acquisition with three different motion states. The notable acquisition parameters were: FOV=224×180×150 mm³, 1.5-mm isotropic resolution, matrix size=150×120×100×48×24 (k_{y}×k_{z}×kₓ×nEcho×nTI), the last 4 TIs were navigator acquisition with FA=10°, FA of GE=30°, ESP=0.7 ms, TE range of each readout=1.3-34.2 ms, TR=2.1 s, 57 TRs were acquired to form 4 radial-block lines and the acquisition time of each dataset was 2 minutes. The block size of the spatiotemporal CAIPI encoding was 10× 8 (k_{y}× k_{z}), corresponding to a 80× undersampling in the k-t space, and 57 blocks were acquired for each TI with a 4-line golden angle radial block-wise pattern that provides another 4× acceleration, resulting in an overall undersampling rate of 320× in the k-t space. The k-t calibration scan was acquired to estimate the B₀ and coil sensitivity maps for each dataset using a GE sequence with bipolar readout, where data were acquired with the same FOV and echo spacing as the imaging scan. Other acquisition parameters were: matrix size=42× 32× 150×7 (k_{y}×k_{z}×kₓ×nEcho), TR=22 ms. The k-space center (12×12) was fully-sampled and the rest of k-space was undersampled along k_{y} and k_{z} by a factor of 2×2, resulting in a total acquisition time of ˜10 s. GRAPPA was used to reconstruct the missing data points in the k-t calibration data, where the central fully-sampled calibration data were used to calibrate the GRAPPA kernels.

The motion-corrupted data were synthesized by combining the 1-18 TRs of the 1^{st} dataset, 18-38 TRs of the 2^{nd} dataset, and 39-57 TRs of the 3rd dataset. The 4D navigators were also extracted from the corresponding TRs of the 3 datasets to estimate the 3D rigid motion and ΔB₀ of each TR. In addition to reconstructing the synthesized motion-corrupted data using the proposed motion-corrected subspace reconstruction with estimated parameters, datasets at the 3 head positions were also reconstructed to obtain reference motion parameters and ΔB₀s, and the quantitative maps of the 1^{st} position is used as reference.

The subspace reconstruction techniques described above were was used for both navigator and image reconstruction. For the navigator reconstruction, the subspace bases were generated using principal component analysis (“PCA”) from the simulated signals with different T₂* decays and ΔB₀s, ranging from 5 ms to 400 ms and −50 Hz to 50 Hz. Six bases were used that can approximate the simulated signal evolutions with an error<1%. For image reconstruction, 8 bases were extracted from the simulated IR-GE signal evolutions (error<0.2%) with the EPG method, with a wide range of quantitative parameters:: T₁ from 400 ms to 5000 ms, T₂* from 5 ms to 500 ms, Bit factor from 0.75 to 1.25. The subspace reconstruction was solved by the alternating direction method of multipliers (ADMM) algorithm, and a maximum number of iterations=100 was set as the stop criterion, with a lambda of 0.01. The dictionary for quantitative parameter fitting was generated with the same parameter range in the basis generation of the IR-GE sequence. After dictionary matching, T₁, T₂*, PD, B₁+ can be obtained, the estimated Bit maps were fitted spatially by a 2^{nd}-order polynomial function to remove residual artifacts, since the Bit field should be smooth in the spatial domain.

The 3D rigid motion between navigators was estimated by FLIRT using the echo-averaged volumes, to obtain motion parameters (6 DOFs) for each TR. Freesurfer was used to auto-segment 159 ROIs using the synthesized T₁-weighted image of the motion-free data for the ROI analysis in the prospective in-vivo experiment, including cortical, subcortical, white matter and cerebellum regions after removing ROIs smaller than 200 voxels. Before segmentation, the different image volumes acquired from stationary and motion scans were co-registered by FLIRT, and the skull was removed by BET.

The systems and methods described in the present disclosure provide a motion correction technique for 3D-EPTI, and other spatiotemporal acquisitions, using an efficient 4D navigator acquisition with motion-corrected subspace reconstruction. Continuous and accurate tracking of 3D subject motion and B₀ changes at every TR (e.g., ˜2 s) are provided by the 4D navigator, at a cost to the SNR efficiency of less than 1%. The estimated motion and ΔB₀ parameters are modeled and incorporated into the 3D subspace reconstruction, allowing fast and motion-robust quantitative neuroimaging using 3D-EPTI.

The proposed 4D navigator acquisition is designed with several optimizations to achieve high efficiency. The optimized spatiotemporal acquisition not only provides data at multiple echoes to estimate the field change, but also enables a large k-space coverage after each excitation. Comparing to multi-echo GRE acquisition, the continuous readouts requires fewer RF excitations, thereby reducing the impact of navigator acquisition on the signal recovery. The use of a smaller FA in the navigator acquisition facilitates minimal cost in SNR efficiency (e.g., less than 1%) while providing sufficient SNR in the low resolution navigator to accurately estimate motion parameters and B₀ changes. An asymmetric partial Fourier scheme in the navigator acquisition can be employed in some embodiments to allow a larger coverage with fewer excitations (e.g., only four excitations). FIG. 2C shows an example partial Fourier scheme with four excitations, which can provide accurate estimation with close accuracy to the nine-excitation case, which can also be useful in the design of other types of navigators. The ability of the proposed 4D navigator to accurately track motion and ΔB₀ was described above with respect to an IR-GE 3D-EPTI, but can also be readily applied to other sequences (e.g., GRASE 3D-EPTI, other EPTI-based sequences) to provide efficient motion navigation.

Aspects of the present disclosure may contribute to an efficient approach to correct 2D/3D motion in spatiotemporal time-resolving MRI, to avoid detrimental image artifacts due to subject movements. Example implementations of the proposed motion estimation method acquires motion navigators efficiently for the spatiotemporal sequences, which does not require additional hardware and do not or only need minimal modification to the original acquisition. By modeling motion using the estimated parameters, the modeling error of reconstruction due to motion can be corrected, which can effectively remove the motion artifacts and blurring in the reconstructed images.

Spatiotemporal time-resolving MR imaging techniques may be developed and implemented, for example, for fast multi-contrast and quantitative mapping. The self-navigated estimation and motion-corrected reconstruction techniques described in the present disclosure may be used for spatiotemporal techniques, such as EPTI-based techniques. Aspects of the present disclosure may help improve the robustness of spatiotemporal time-resolving MR imaging to subject motion and facilitate clinical and neuroscience applications.

Referring particularly now to FIG. 5, an example of a magnetic resonance imaging (“MRI”) system 500 that can implement the methods described here is illustrated. The MRI system 500 includes an operator workstation 502 that may include a display 504, one or more input devices 506 (e.g., a keyboard, a mouse), and a processor 508. The processor 508 may include a commercially available programmable machine running a commercially available operating system. The operator workstation 502 provides an operator interface that facilitates entering scan parameters into the MRI system 500. The operator workstation 502 may be coupled to different servers, including, for example, a pulse sequence server 510, a data acquisition server 512, a data processing server 514, and a data store server 516. The operator workstation 502 and the servers 510, 512, 514, and 516 may be connected via a communication system 540, which may include wired or wireless network connections.

The pulse sequence server 510 functions in response to instructions provided by the operator workstation 502 to operate a gradient system 518 and a radiofrequency (“RF”) system 520. Gradient waveforms for performing a prescribed scan are produced and applied to the gradient system 518, which then excites gradient coils in an assembly 522 to produce the magnetic field gradients Gₓ, G_{y}, and G_{z} that are used for spatially encoding magnetic resonance signals. The gradient coil assembly 522 forms part of a magnet assembly 524 that includes a polarizing magnet 526 and a whole-body RF coil 528.

RF waveforms are applied by the RF system 520 to the RF coil 528, or a separate local coil to perform the prescribed magnetic resonance pulse sequence. Responsive magnetic resonance signals detected by the RF coil 528, or a separate local coil, are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the whole-body RF coil 528 or to one or more local coils or coil arrays.

The RF system 520 also includes one or more RF receiver channels. An RF receiver channel includes an RF preamplifier that amplifies the magnetic resonance signal received by the coil 528 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 510 may receive patient data from a physiological acquisition controller 530. By way of example, the physiological acquisition controller 530 may receive signals from a number of different sensors connected to the patient, including electrocardiogram (“ECG”) signals from electrodes, or respiratory signals from a respiratory bellows or other respiratory monitoring devices. These signals may be used by the pulse sequence server 510 to synchronize, or “gate,” the performance of the scan with the subject's heart beat or respiration.

The pulse sequence server 510 may also connect to a scan room interface circuit 532 that receives signals from various sensors associated with the condition of the patient and the magnet system. Through the scan room interface circuit 532, a patient positioning system 534 can receive commands to move the patient to desired positions during the scan.

The digitized magnetic resonance signal samples produced by the RF system 520 are received by the data acquisition server 512. The data acquisition server 512 operates in response to instructions downloaded from the operator workstation 502 to receive the real-time magnetic resonance data and provide buffer storage, so that data is not lost by data overrun. In some scans, the data acquisition server 512 passes the acquired magnetic resonance data to the data processor server 514. In scans that require information derived from acquired magnetic resonance data to control the further performance of the scan, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520 or the gradient system 518, or to control the view order in which k-space is sampled. For example, the data acquisition server 512 may acquire magnetic resonance data and processes it in real-time to produce information that is used to control the scan.

The data processing server 514 receives magnetic resonance data from the data acquisition server 512 and processes the magnetic resonance data in accordance with instructions provided by the operator workstation 502. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images. For example, the data processing server 514 may implement the motion estimation and/or motion correction techniques described in the present disclosure.

Images reconstructed by the data processing server 514 are conveyed back to the operator workstation 502 for storage. Real-time images may be stored in a data base memory cache, from which they may be output to operator display 502 or a display 536. Batch mode images or selected real time images may be stored in a host database on disc storage 538. When such images have been reconstructed and transferred to storage, the data processing server 514 may notify the data store server 516 on the operator workstation 502. The operator workstation 502 may be used by an operator to archive the images, produce films, or send the images via a network to other facilities.

The MRI system 500 may also include one or more networked workstations 542. For example, a networked workstation 542 may include a display 544, one or more input devices 546 (e.g., a keyboard, a mouse), and a processor 548. The networked workstation 542 may be located within the same facility as the operator workstation 502, or in a different facility, such as a different healthcare institution or clinic.

The networked workstation 542 may gain remote access to the data processing server 514 or data store server 516 via the communication system 540. Accordingly, multiple networked workstations 542 may have access to the data processing server 514 and the data store server 516. In this manner, magnetic resonance data, reconstructed images, or other data may be exchanged between the data processing server 514 or the data store server 516 and the networked workstations 542, such that the data or images may be remotely processed by a networked workstation 542.

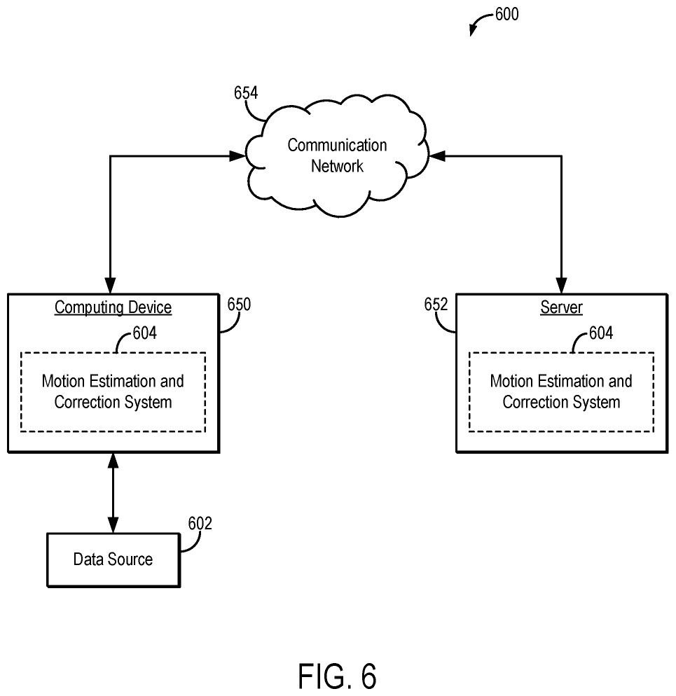

Referring now to FIG. 6, an example of a system 600 for motion estimation and correction in accordance with some embodiments of the systems and methods described in the present disclosure is shown. As shown in FIG. 6, a computing device 650 can receive one or more types of data (e.g., k-space data, k-t space data) from data source 602, which may be a magnetic resonance imaging data source. In some embodiments, computing device 650 can execute at least a portion of a motion estimation and correction system 604 to estimate and retrospectively correct motion from data received from the data source 602.

Additionally or alternatively, in some embodiments, the computing device 650 can communicate information about data received from the data source 602 to a server 652 over a communication network 654, which can execute at least a portion of the motion estimation and correction system 604. In such embodiments, the server 652 can return information to the computing device 650 (and/or any other suitable computing device) indicative of an output of the motion estimation and correction system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data. For example, the computing device 650 and/or server 652 can reconstruct motion-corrected images from k-space and/or k-t space data received from the data source 602.

In some embodiments, data source 602 can be any suitable source of data (e.g., k-space data, k-t space data, images reconstructed from k-space and/or k-t space data), such as an MRI system, another computing device (e.g., a server storing k-space and/or k-t space data), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

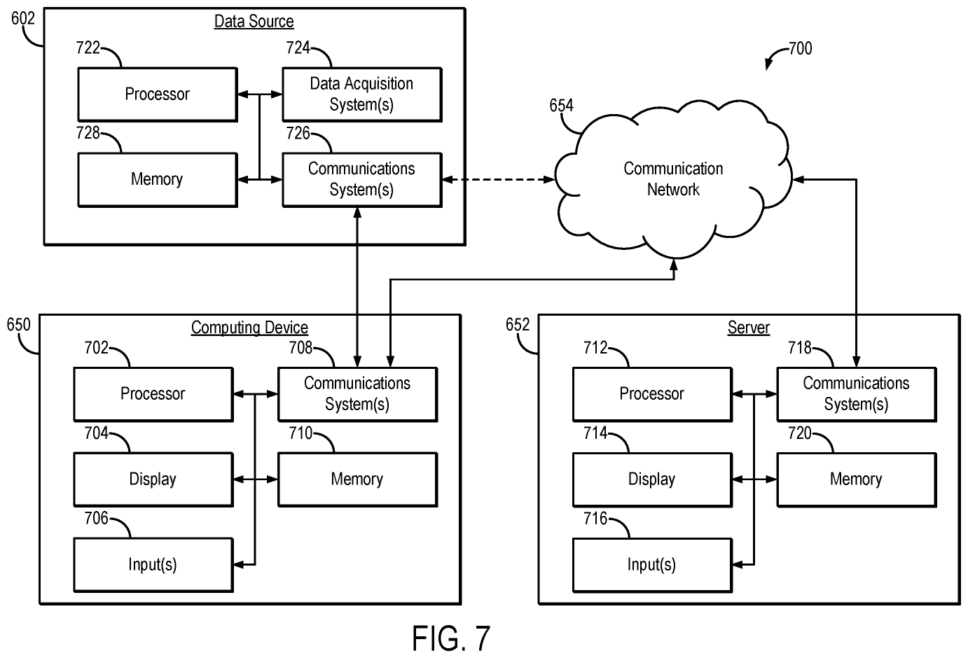

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method of FIG. 3, the method of FIG. 4).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the method of FIG. 3, the method of FIG. 4).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include an MRI system. Additionally or alternatively, in some embodiments, the one or more data acquisition systems 724 can include any suitable hardware, firmware, and/or software for coupling to and/or controlling operations of an MRI system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 702. In such embodiments, processor 722 can execute at least a portion of the program to generate images, transmit information and/or content (e.g., data, images) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for magnetic resonance imaging, the method comprising:
(a) accessing magnetic resonance data acquired with a magnetic resonance imaging (MRI) system, wherein the magnetic resonance data comprise spatiotemporally acquired data and navigator data;
(b) estimating motion data from the navigator data using a computer system, wherein the motion data comprise motion parameters associated with subject motion that occurred when the magnetic resonance data were acquired and B₀ change data that indicate B₀ inhomogeneity changes caused by the subject motion;
(c) reconstructing an image from the spatiotemporally acquired data using a subspace reconstruction framework that models motion using the motion data, wherein the image has reduced motion artifacts.

2. The method of claim 1, wherein the motion data are estimated from navigator images reconstructed from the navigator data using a second subspace reconstruction framework.

3. The method of claim 2, wherein estimating the motion data comprises:
reconstructing the navigator images from the navigator data using the second subspace reconstruction framework;
estimating the motion parameters from the navigator images; and
estimating the B₀ change data by applying the motion parameters to a pre-estimated B₀ map.

4. The method of claim 2, wherein the second subspace reconstruction framework models temporal subspace bases generated based on a signal model using an extended phase graph simulation.

5. The method of claim 2, wherein the second subspace reconstruction framework models subspace bases based on a principal component analysis of simulated signals with different signal parameters.

6. The method of claim 5, wherein the different signal parameters comprise at least one of different T2* decay values or different B₀ change (ΔB₀) values.

7. The method of claim 6, wherein the different T2* decay values are selected from a range of 5 ms to 400 ms.

8. The method of claim 6, wherein the different B₀ change values are selected from a range of −50 Hz to +50 Hz.

9. The method of claim 1, wherein the subspace reconstruction framework models subspace bases generated based on a signal model using an extended phase graph simulation with different signal parameters.

10. The method of claim 9, wherein the different signal parameters comprise at least one of different T1 values, different T2* values, or different B1+ factors.

11. The method of claim 10, wherein the different T1 values are selected from a range of 400 ms to 5000 ms.

12. The method of claim 10, wherein the different T2* values are selected from a range of 5 ms to 500 ms.

13. The method of claim 10, wherein the different B1+ factors are selected from a range of 0.75 to 1.25.

14. The method of claim 1, wherein the magnetic resonance data are acquired using an echo planar time-resolved imaging (EPTI) based pulse sequence.

15. The method of claim 14, wherein the EPTI-based pulse sequence is an inversion recovery pulse sequence having a data acquisition period and a magnetization recovery period, wherein the spatiotemporally acquired data are acquired during the data acquisition period and the navigator data are acquired during the magnetization recovery period.
